(19) United States
(12) Patent Application Publication
(10) Pub. No.: US 2023/0392217 A1
(43) Pub. Date: Dec. 7, 2023

(54) COMPOSITION FOR DIAGNOSING SUSCEPTIBILITY OF STAPHYLOCOCCUS AUREUS TO ANTIBIOTICS, AND USE THEREOF

(71) Applicant: INSTITUT PASTEUR KOREA, Seongnam-si, Gyeonggi-do (KR)

(72) Inventors: Soojin JANG, Suwon-si, Gyeonggi-do (KR); Yunmi LEE, Yongin-si, Gyeonggi-do (KR)

(73) Assignee: INSTITUT PASTEUR KOREA, Seongnam-si, Gyeonggi-do (KR)

(21) Appl. No.: 18/034,064

(22) PCT Filed: Oct. 15, 2021
(86) PCT No.: PCT/KR2021/014293

§ 371 Date: Apr. 27, 2023

(30) Foreign Application Priority Data
Oct. 29, 2020 (KR) 10-2020-0142045
Oct. 1, 2021 (KR) 10-2021-0130787

(51) Int. Cl. C12Q 1/689; G01N 33/569; C07K 16/12

(52) U.S. Cl. CPC C12Q 1/689; G01N 33/56938; C07K 16/1271; C12Q 2600/106; C12Q 2600/158; G01N 2800/52

(57) ABSTRACT

The present invention relates to a composition for diagnosing the susceptibility of Staphylococcus aureus to antibiotics, and a use thereof. Particularly, it has been ascertained that sosA is a marker specific to Staphylococcus aureus, and that the expression level of sosA is exhibited similarly to antibiotic susceptibility/resistance properties so as to be usable as a means for diagnosing the susceptibility/resistance of Staphylococcus aureus to antibiotics, and the present invention diagnoses the susceptibility/resistance of Staphylococcus aureus present in samples to antibiotics by using antibodies specifically binding to protein SosA, and thus provides, as a means for diagnosing the susceptibility of Staphylococcus aureus to antibiotics, a composition containing an agent for measuring the mRNA expression level of the sosA gene or an agent for measuring the expression level of the SosA protein.

### TECHNICAL FIELD

The present disclosure relates to a composition for diagnosing susceptibility of Staphylococcus aureus to antibiotics and a use thereof.

### BACKGROUND ART

Staphylococcus aureus is a pathogen detected in about 30% of the population and known as a problematic pathogen that causes nosocomial infection. Staphylococcus aureus is the most problematic pathogen worldwide because it causes infection in the skin and soft tissues to develop pyoderma and also causes serious life-threatening systemic infection such as osteomyelitis, endocarditis, sepsis, and bacteremia.

The most commonly used method to prevent infection by pathogens and treat infectious diseases is to use antibiotics such as penicillin and methicillin, but pathogens that show resistance to antibiotics are being developed due to excessive use of antibiotics. Since the first report of Staphylococcus aureus that shows resistance to the antibiotic methicillin in the United Kingdom in 1961, the discovery of pathogens showing resistance to various antibiotics has increased worldwide.

Pathogens acquire resistance to antibiotics by mutation or acquisition of antibiotic-resistant genes as a means of self-defense, and the frequency of occurrence of such resistant bacteria is rising with an increase in the misuse and abuse of antibiotics. In addition, an increase in antibiotic-resistant pathogens is due not only to nosocomial infections, but also to the development of transportation and indiscriminate use of antibiotics. The number of patients infected with antibiotic-resistant pathogens is gradually increasing worldwide. Therefore, for efficient control and treatment of pathogen infection, selection of treatment agents based on accurate and rapid diagnosis of antibiotic resistance of pathogens is crucial.

The present inventors intended to determine that SosA, a cell differentiation regulatory protein of Staphylococcus aureus, a representative pathogen, appears to have a specific expression pattern in response to antibiotics and provide a novel diagnostic composition and diagnostic method capable of determining susceptibility/resistance to antibiotics using the same.

### PRIOR ART DOCUMENT

### Patent Document

Korean Patent No. 10-2124614

### DISCLOSURE OF THE INVENTION

### Technical Goals

The present disclosure relates to a composition for diagnosing susceptibility of Staphylococcus aureus to antibiotics and a use thereof, and specifically, an object of the present disclosure is to provide a composition including a preparation for measuring an mRNA expression level of a sosA gene or a preparation for measuring an expression level of a SosA protein as a means to diagnose susceptibility of Staphylococcus aureus to antibiotics, by identifying that sosA is a marker specific to Staphylococcus aureus, determining that an expression level of sosA is closely related to antibiotic susceptibility/resistance properties so as to be used as a means to diagnose susceptibility/resistance of Staphylococcus aureus to antibiotics, and developing antibodies that specifically bind to the SosA protein to diagnose antibiotic sensitivity/resistance of Staphylococcus aureus present in a sample.

### Technical Solutions

The present disclosure provides a composition for diagnosing susceptibility of Staphylococcus aureus to an antibiotic, including a preparation for measuring an mRNA expression level of a sosA gene or a preparation for measuring an expression level of a SosA protein.

In addition, the present disclosure provides a kit for diagnosing susceptibility of Staphylococcus aureus to an antibiotic, including the composition.

In addition, the present disclosure provides an antibody for diagnosing susceptibility of Staphylococcus aureus, which binds to an antigen having an amino acid sequence represented by SEQ ID NO: 7.

In addition, the present disclosure provides a method of diagnosing susceptibility of Staphylococcus aureus to an antibiotic, including treating a biological sample isolated from a patient infected with Staphylococcus aureus with the antibiotic and an antibody that specifically binds to a SosA protein to detect Staphylococcus aureus having the SosA protein expressed on its surface; and determining that the Staphylococcus aureus having the SosA protein expressed on its surface has susceptibility to the antibiotic.

In addition, the present disclosure provides a method of screening antibiotics against Staphylococcus aureus, including treating Staphylococcus aureus with a candidate material to measure an mRNA expression level of a sosA gene or an expression level of a SosA protein; and determining that the candidate material is an antibiotic exhibiting antimicrobial activity against Staphylococcus aureus when the mRNA expression level of the sosA gene or an expression level of the SosA protein of Staphylococcus aureus increases.

In addition, the present disclosure provides a method of providing information for predicting the prognosis of antibiotic treatment for infection with Staphylococcus aureus, including administering an antibiotic to a subject and measuring an mRNA expression level of a sosA gene or an expression level of a SosA protein in a biological sample isolated from the subject.

In addition, the present disclosure provides an antibody for diagnosing susceptibility of Staphylococcus aureus to an antibiotic, wherein the antibody specifically binds to a SosA protein.

### Advantageous Effects

According to the present disclosure, by identifying that sosA is a marker specific to Staphylococcus aureus, determining that a sosA expression level is closely related to antibiotic susceptibility/resistance properties to be used as a means to diagnose susceptibility/resistance of Staphylococcus aureus to antibiotics, and diagnosing antibiotic susceptibility/resistance of Staphylococcus aureus present in a sample using antibodies that specifically bind to a SosA protein, a composition including a preparation for measuring an mRNA expression level of a sosA gene or a preparation for measuring an expression level of a SosA protein may be provided as a means to diagnose susceptibility of Staphylococcus aureus to antibiotics and a method capable of quickly and accurately predicting an effect of antibiotics in antibiotic treatment for patients infected with Staphylococcus aureus.

### BRIEF DESCRIPTION OF DRAWINGS

FIG. 1 shows a result of detecting 16s rRNA and a sosA gene in various microorganisms to detect Staphylococcus aureus.

FIG. 2 shows results of analyzing RNA expression of sosA upon antibiotic treatment in methicillin-sensitive S. aureus (MSSA).

FIG. 3 shows results of analyzing RNA expression of sosA upon antibiotic treatment in methicillin-resistant S. aureus (MRSA).

FIG. 4 is a diagram showing an experimental method capable of accurately and quickly predicting an effect of antibiotics administered for a therapeutic purpose using sosA biomarkers in an animal model.

FIG. 5 shows a result of analyzing a sosA expression level of Staphylococcus aureus following antibiotic administration in an animal model.

FIG. 6 shows a result of analyzing a survival rate of an animal model following antibiotic administration in an animal model.

FIG. 7 shows a result of detecting a SosA protein by mRNA expression of sosA using the entire SosA protein sequence, the protein sequence used for SosA-specific antibody development, and a developed antibody.

FIG. 8 is a diagram showing a method of using SosA antibodies to diagnose antibiotic-susceptible/resistant pathogen.

FIG. 9 shows a result of detecting a pathogen that expresses SosA without a lysis process of bacteria in a sample using SosA antibodies.

FIG. 10 shows a result of identifying SosA expression of Staphylococcus aureus according to concentration of ciprofloxacin using SosA antibodies.

FIG. 11 shows results of verifying an antigen-antibody response of seven types of SosA antibodies using Western blot.

### BEST MODE FOR CARRYING OUT THE INVENTION

The terms used herein have been selected from currently widely used general terms as much as possible in consideration of functions herein, but these may vary depending on the intentions or precedents of those skilled in the art, the emergence of new technologies, and the like. In addition, in specific cases, there are terms arbitrarily selected by the applicant, and in this case, the meaning will be described in detail in the description of the disclosure. Therefore, the terms used herein should not be defined as simple names of terms, but based on the meaning of the term and the overall contents of the present disclosure.

Unless otherwise defined, all terms used herein, including technical or scientific terms, have the same meaning as commonly understood by those skilled in the art to which the present disclosure pertains. Terms such as those defined in commonly used dictionaries should be construed as having meanings consistent with the meaning in the context of the relevant art and are not to be construed in an ideal or overly formal meaning unless clearly defined in the present application.

A numerical range includes a numerical value defined in the range. All maximum numerical limits given herein include all lower numerical limits as clearly stated on the lower numerical limits. All minimum numerical limits given herein include all higher numerical limits as clearly stated on the higher numerical limits. All numerical limits given herein will include all better numerical ranges within a wider numerical range as clearly stated on narrower numerical limits.

Unless otherwise indicated, a direction of nucleic acid sequences is read from left to right, that is, from 5′ to 3′, respectively, and a direction of amino acid sequences is read from left to right, that is, from an amino group to a carboxyl group.

Hereinafter, the present disclosure will be described in more detail.

The present disclosure provides a composition for diagnosing susceptibility of Staphylococcus aureus to an antibiotic, including a preparation for measuring an mRNA expression level of a sosA gene or a preparation for measuring an expression level of a SosA protein.

The SosA protein is a cell differentiation regulatory protein expressed on a surface of Staphylococcus aureus. Specifically, the SosA protein has an amino acid sequence represented by SEQ ID NO: 1 and is encoded in a sosA gene, wherein the sosA gene has a nucleotide sequence represented by SEQ ID NO: 2.

The antibiotic may be ciprofloxacin, vancomycin, methicillin, tetracycline, amikacin, kanamycin, gentamicin, streptomycin, doxycycline, tigecycline, erythromycin, azithromycin, clarithromycin, clindamycin, linezolid, rifampicin, levofloxacin, or moxifloxacin, but is not limited thereto.

The preparation for measuring the mRNA expression level of the sosA gene is an antisense oligonucleotide, primer pair, or probe that specifically binds to an mRNA of the sosA, and the preparation for measuring the expression level of the SosA protein is an oligopeptide, monoclonal antibody, polyclonal antibody, chimeric antibody, ligand, peptide nucleic acid (PNA), or aptamer that specifically binds to the SosA protein.

In addition, the present disclosure provides a kit for diagnosing susceptibility of Staphylococcus aureus to an antibiotic, including the composition.

The kit may be an RT-PCR kit, a competitive RT-PCR kit, a real-time RT-PCR kit, a digital PCR kit, a DNA chip kit, or a protein chip kit.

In addition, the present disclosure provides an antibody for diagnosing susceptibility of Staphylococcus aureus. The antibody specifically binds to an antigen having an amino acid sequence represented by SEQ ID NO: 7.

In addition, the present disclosure provides a method of diagnosing susceptibility of Staphylococcus aureus to an antibiotic, including detecting Staphylococcus aureus having a SosA protein expressed on its surface by treating a biological sample isolated from a patient infected with Staphylococcus aureus with the antibiotic and an antibody that specifically binds to the SosA protein; and determining that the Staphylococcus aureus having the SosA protein expressed on its surface has susceptibility to the antibiotic.

The antibody that specifically binds to the SosA protein binds to the antigen having an amino acid sequence represented by SEQ ID NO: 7.

The detecting of Staphylococcus aureus having the SosA protein expressed on its surface includes further treating with a fluorescent material conjugated with a secondary antibody that specifically binds to the antibody that specifically binds to the SosA protein. The fluorescent material may be a green fluorescent protein (GFP), yellow fluorescent protein (YFP), blue fluorescent protein (BFP), or red fluorescent protein (RFP).

In addition, the present disclosure provides a method of screening antibiotics against Staphylococcus aureus, including treating Staphylococcus aureus with a test material to measure an mRNA expression level of a sosA gene or an expression level of a SosA protein; and determining that the test material is an antibiotic exhibiting antimicrobial activity against Staphylococcus aureus when the mRNA expression level of the sosA gene or the expression level of the SosA protein of Staphylococcus aureus increases.

The mRNA expression level of the sosA gene may be measured using reverse transcriptase polymerase reaction (RT-PCR), competitive RT-PCR, real time quantitative RT-PCR, RNase protection method, Northern blotting, or DNA chip technology, and the expression level of the SosA protein may be measured using Western blotting, enzyme linked immunosorbent assay (ELISA), radioimmunoassay (RIA), radial immunodiffusion, Ouchterlony immunodiffusion, rocket immunoelectrophoresis, immunohistochemical staining, immunoprecipitation assay, complement fixation assay, immunofluorescence, immunochromatography, fluorescence activated cell sorter (FACS) analysis, or protein chip technology, but are not limited thereto.

The test material refers to an unknown candidate material used in screening to test whether it affects the expression amount of a gene or affects expression or activity of a protein. The sample includes chemicals, nucleotides, antisense-RNA, small interference RNA (siRNA), and natural product extracts, but is not limited thereto.

In addition, the present disclosure provides a method of providing information for predicting the prognosis of antibiotic treatment for infection with Staphylococcus aureus, including administering an antibiotic to a subject and measuring an mRNA expression level of a sosA gene or an expression level of a SosA protein in a biological sample isolated from the subject.

The subject may be a patient, companion animal, or domestic animal infected with Staphylococcus aureus, and the biological sample may be blood, body fluid, saliva, and urine separated from the subject.

In addition, the present disclosure provides an antibody for diagnosing susceptibility of Staphylococcus aureus to an antibiotic, wherein the antibody specifically binds to a SosA protein.

The antibody may include a heavy chain having an amino acid sequence represented by SEQ ID NO: 8 and a light chain having an amino acid sequence represented by SEQ ID NO: 9, the antibody may include a heavy chain having an amino acid sequence represented by SEQ ID NO: 10 and a light chain having an amino acid sequence represented by SEQ ID NO: 11, the antibody may include a heavy chain having an amino acid sequence represented by SEQ ID NO: 12 and a light chain having an amino acid sequence represented by SEQ ID NO: 13, the antibody may include a heavy chain having an amino acid sequence represented by SEQ ID NO: 14 and a light chain having an amino acid sequence represented by SEQ ID NO: 15, the antibody may include a heavy chain having an amino acid sequence represented by SEQ ID NO: 16 and a light chain having an amino acid sequence represented by SEQ ID NO: 17, the antibody may include a heavy chain having an amino acid sequence represented by SEQ ID NO: 18 and a light chain having an amino acid sequence represented by SEQ ID NO: 19, and the antibody may include a heavy chain having an amino acid sequence represented by SEQ ID NO: 20 and a light chain having an amino acid sequence represented by SEQ ID NO: 21.

### MODES FOR CARRYING OUT THE INVENTION

Hereinafter, experimental examples and example embodiments will be described in detail to help the understanding of the present disclosure. However, the following experimental examples and example embodiments are merely illustrative of the content of the present disclosure, and the scope of the present disclosure is not limited to the following experimental examples and example embodiments. The experimental examples and example embodiments of the present disclosure are provided to more completely explain the present disclosure to those skilled in the art.

### <Experimental Example> Experimental Materials and Methods

The following experimental examples are intended to provide experimental examples commonly applied to each example embodiment according to the present disclosure.

1. Microorganisms

Strains used in the experiment were Acinetobacter baumannii (A. baumannii), Escherichia coli K12 (E. coli K12), Pseudomonas aeruginosa (P. aeruginosa), Streptococcus pneumoniae (S. pneumoniae), and Staphylococcus aureus (S. aureus).

The microorganisms to be used in the experiment were inoculated in fresh Mueller Hinton media and cultured under an aerobic environment at 37° C.

2. Extraction of RNA

Each centrifuged strain was washed with 350 ul of RNA protect bacteria reagent. The pellets were centrifuged at 13,000 rpm for 2 minutes and suspended in 220 ul of EDTA buffer (a solution mixed with 200 ul of 50 mM EDTA, 10 ul of 10 mg/ml lysozyme, and 10 ul of 5 mg/ml lysostaphin). Culture was performed at 37° C. for 30 minutes, and then sonication was followed 10 times for 30 seconds each. Each sample was enzymatically and mechanically lysed, and then RNA was extracted using an RNeasy Mini Kit (Qiagen) according to the manufacturer's instructions. Total RNA was eluted with RNase-free ddH₂O. RNA was diluted to a concentration of 50 ng/ul, and remaining genomic DNA was removed using DNase I (Sigma Aldrich). The reaction volume was set to 10 ul, consisting of 1 ul of buffer solution, 1 ul of DNase I stock solution, and 5 ul of 50 ng/ul RNA. The mixture was cultured at room temperature for 15 minutes, and the reaction was discontinued by adding 40 ul of RNase-free purified water.

3. Real-Time Quantitative PCR

The RNA extracted from each strain was investigated for expression of a sosA gene using a primer set in Table 1 below.

| Gene | | Sequences | SEQ ID NO. |
|---|---|---|---|
| _{SOS}A | F | 5′ CGGAACAAGTGTACGAAATG 3′ | 3 |
| | R | 5′ GGGTCTTTATGTTCGTATGC 3′ | 4 |
| tRNA | F | 5′ GATGATTGAAGGGGAAATGG 3′ | 5 |
| | R | 5′ GGTGTCGCAACTTTTTCAAG 3′ | 6 |

For PCR reaction, a volume of a PCR mixture was set to 25 μL in total to include 5 ul of 5 ng/ul RNA, 1 ul of each primer, 0.25 ul of RT enzyme, and 12.5 ul of SYBR Green I PCR master mix (Qiagne). After reverse transcription at 50° C. for 10 minutes using Applied Biosystems ViiA 7, initiation at 95° C. for 5 minutes, denaturation at 95° C. for 10 seconds, and annealing at 58° C. for 10 seconds and extension at 72° C. for 20 seconds were performed for 40 cycles. Fluorescence acquisition was then performed at 72° C. at the end of elongation. The threshold cycle (Ct) value was calculated by determining a point at which the threshold limit of fluorescence was exceeded. In order to find out whether primer-dimer and nonspecific bonds were formed at termination of PCR, the melting curve was set at a ramp rate of 0.1° C./sec. To analyze the gene expression level, mean Ct for reference gene tRNA was subtracted from mean Ct for sosA (ΔCt). ΔΔCt was then used to determine the multiples of expression changes.

4. Western Blot

After collecting microorganisms, the pellets were suspended in 300 ul of lysis buffer (50 mM Tris/Hcl, pH7.5, 145 mM NaCl, 4 mM EDTA, pH8.0, 100 ug/ml Lysostaphin, and 1× proteinase inhibitor), cultured at 37° C. for 1 hour, and sonicated 20 times for 30 seconds each. The concentration of total protein was determined using the Bradford colorimetric method and set based on BSA. The same amount of protein was loaded into a 15% polyacrylamide-SDS gel and transferred to a polyvinylidene fluoride (PVDF) membrane. Blot was blocked with 5% fat-free milk powder in DPBS, and SosA-specific antibody was diluted at 1:3,000 to be treated overnight at 4° C. The treated membrane was washed three times with TBS-T (20 mM Tris, 137 mM NaCl, 0.1% Tween 20) at room temperature for 1 hour. Thereafter, goat anti-mouse peroxidase-bound secondary antibody (Abcam) was treated at 1:6,000 for 1 hour in 5% fat-free milk powder, followed by washing three times in the same manner as above. Immune response bands were detected with the ECL detection kit (Pierce) using a LuminoGraph II instrument.

5. Measurement of Fluorescence

Pellets of microorganisms were suspended with 1 ml of DPBS containing sosA-specific antibodies that were diluted at 1:2,000 and cultured overnight at 37° C., 180 rpm. Thereafter, centrifugation was performed at 13,000 rpm for 1 minute, secondary antibodies (anti-mouse FITC, Abcam) diluted at 1:500 were treated, and culture was performed at 37° C., 180 rpm for 3 hours. 100 ul of a sample was transferred to a 12-well plate containing an 18 mm cover glass and centrifuged at 1,100 rpm for 1 minute, and the bacteria were attached to a cover glass. The plates were washed three times with DPBS, and centrifugation was performed at 1,100 rpm for 1 minute. The cover glass was attached to the slide, and then imaging was performed using the Zeiss LSM 5 live confocal microscope. Images were acquired using Plan-Apochromat 63×1.4 NA objectives, and analysis was performed to detect GFP fluorescence by setting to 488 nm for excitation wavelength and 513±12 nm for emission wavelength.

In addition, each sample was transferred to a 96-well plate, and absorbance measurement and fluorescence analysis were performed using Ensight (PerkinElmer). In the case of green fluorescence, the excitation wavelength was at 488 nm and the emission wavelength was at 528 nm, and in the case of red fluorescence, the excitation wavelength was at 558 nm and the emission wavelength was at 615 nm. The standard curve for signal intensity was used with two times dilution of the sample regardless of fixation.

### Example 1. Detection of Staphylococcus aureus Using Biomarkers

To determine whether sosA is suitable as a marker for diagnosing antibiotic susceptibility of Staphylococcus aureus, evaluation was performed whether sosA is a specific marker for Staphylococcus aureus. The total genome was extracted from various species of pathogenic bacteria including Acinetobacter baumannii (A. baumannii), Escherichia coli K12 (E. coli K12), Pseudomonas aeruginosa (P. aeruginosa), Streptococcus pneumoniae (S. pneumoniae), and Staphylococcus aureus (S. aureus), followed by analysis on whether the sosA gene is present.

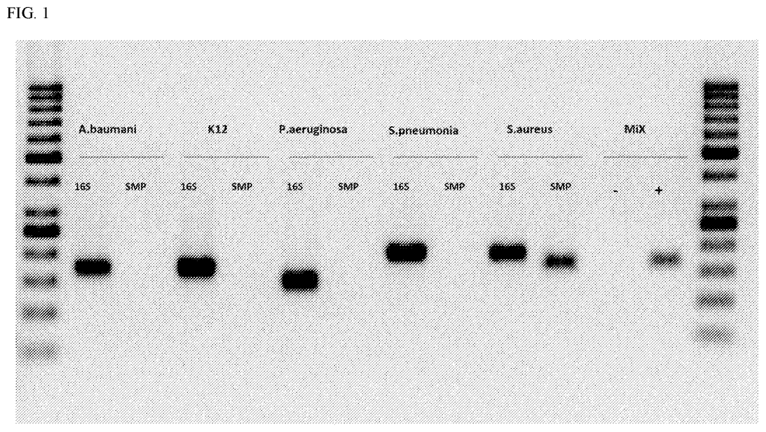

As shown in FIG. 1, 16s rRNA that may prove the presence of each pathogenic bacterium was detected in all bacteria, but the sosA gene was detected only in Staphylococcus aureus or MIX samples including the same. The results demonstrate that sosA is a specific marker for Staphylococcus aureus.

### Example 2. Evaluation on Expression of sosA According to Antibiotic Susceptibility/Resistance

To evaluate whether the susceptibility/resistance upon treatment of various antibiotics in Staphylococcus aureus is related to expression of sosA, the expression level of sosA upon treatment of antibiotics to Staphylococcus aureus with previously known antibiotic susceptibility/resistance was analyzed.

First, methicillin-sensitive S. aureus (MSSA) with known antibiotic susceptibility/resistance properties was treated with ciprofloxacin, vancomycin, methicillin, or tetracycline as shown in Table 2 below, and the expression level of sosA was analyzed.

TABLE 2

| Strain No. | Ciprofloxacin | Vancomycin | Methicillin | Tetracycline |
|---|---|---|---|---|
| MSSA #1 | Susceptible | Susceptible | Susceptible | Susceptible |
| MSSA #2 | Susceptible | Susceptible | Susceptible | Susceptible |
| MSSA #3 | Susceptible | Susceptible | Susceptible | Susceptible |
| MSSA #4 | Susceptible | Susceptible | Susceptible | Susceptible |
| MSSA #5 | Susceptible | Susceptible | Susceptible | Susceptible |
| MSSA #6 | Susceptible | Susceptible | Susceptible | Resistant |
| MSSA #7 | Resistant | Susceptible | Susceptible | Resistant |
| MSSA #8 | Resistant | Susceptible | Susceptible | Resistant |
| MSSA #9 | Susceptible | Susceptible | Susceptible | Resistant |
| MSSA #10 | Susceptible | Susceptible | Susceptible | Susceptible |
| MSSA #11 | Susceptible | Susceptible | Susceptible | Resistant |
| MSSA #12 | Susceptible | Susceptible | Susceptible | Susceptible |
| MSSA #13 | Susceptible | Susceptible | Susceptible | Susceptible |
| MSSA #14 | Susceptible | Susceptible | Susceptible | Susceptible |
| MSSA #15 | Susceptible | Susceptible | Susceptible | Susceptible |
| MSSA #16 | Susceptible | Susceptible | Susceptible | Susceptible |
| MSSA #17 | Susceptible | Susceptible | Susceptible | Susceptible |
| MSSA #18 | Susceptible | Susceptible | Susceptible | Susceptible |
| MSSA #19 | Susceptible | Susceptible | Susceptible | Susceptible |
| MSSA #20 | Resistant | Susceptible | Susceptible | Susceptible |

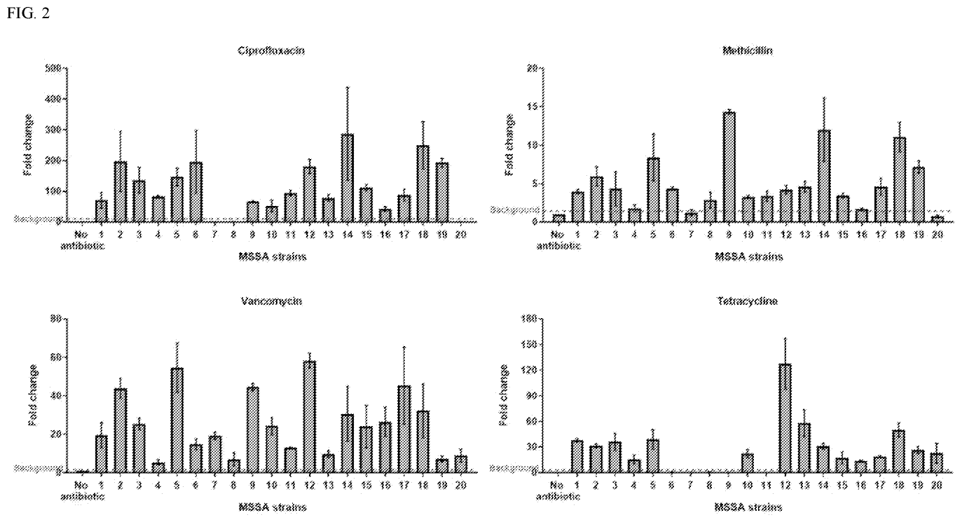

As shown in FIG. 2, the sosA expression level of methicillin-sensitive S. aureus (MSSA) increased when the susceptibility to antibiotics was derived and was inhibited when resistance to antibiotics was derived, wherein the sosA expression level was similar to the previously known antibiotic susceptibility/resistance properties.

In addition, as shown in Table 3 below, methicillin-resistant S. aureus (MRSA) with known antibiotic susceptibility/resistance properties was treated with ciprofloxacin, vancomycin, methicillin, or tetracycline to analyze the expression level of sosA.

TABLE 3

| Strain No. | Ciprofloxacin | Vancomycin | Methicillin | Tetracycline |
|---|---|---|---|---|
| MRSA #1 | Resistant | Susceptible | Resistant | Resistant |
| MRSA #2 | Resistant | Susceptible | Resistant | Resistant |
| MRSA #3 | Susceptible | Susceptible | Resistant | Susceptible |
| MRSA #4 | Susceptible | Susceptible | Resistant | Susceptible |
| MRSA #5 | Resistant | Susceptible | Resistant | Resistant |
| MRSA #6 | Resistant | Susceptible | Resistant | Resistant |
| MRSA #7 | Resistant | Susceptible | Resistant | Susceptible |
| MRSA #8 | Resistant | Susceptible | Resistant | Resistant |
| MRSA #9 | Resistant | Susceptible | Resistant | Susceptible |
| MRSA #10 | Resistant | Susceptible | Resistant | Resistant |
| MRSA #11 | Resistant | Susceptible | Resistant | Resistant |
| MRSA #12 | Resistant | Susceptible | Resistant | Resistant |
| MRSA #13 | Resistant | Susceptible | Resistant | Resistant |
| MRSA #14 | Resistant | Susceptible | Resistant | Resistant |
| MRSA #15 | Resistant | Susceptible | Resistant | Susceptible |
| MRSA #16 | Resistant | Susceptible | Resistant | Susceptible |
| MRSA #17 | Resistant | Susceptible | Resistant | Resistant |
| MRSA #18 | Susceptible | Susceptible | Resistant | Susceptible |
| MRSA #19 | Resistant | Susceptible | Resistant | Resistant |
| MRSA #20 | Resistant | Resistant | Resistant | Resistant |
| MRSA #21 | Resistant | Susceptible | Resistant | Resistant |
| MRSA #22 | Resistant | Susceptible | Resistant | Resistant |
| MRSA #23 | Resistant | Susceptible | Resistant | Resistant |
| MRSA #24 | Susceptible | Susceptible | Susceptible | Susceptible |
| MRSA #25 | Resistant | Susceptible | Resistant | Resistant |
| MRSA #26 | Susceptible | Susceptible | Resistant | Resistant |
| MRSA #27 | Susceptible | Susceptible | Resistant | Susceptible |
| MRSA #28 | Resistant | Susceptible | Resistant | Resistant |
| MRSA #29 | Resistant | Susceptible | Resistant | Resistant |
| MRSA #30 | Resistant | Resistant | Resistant | Resistant |

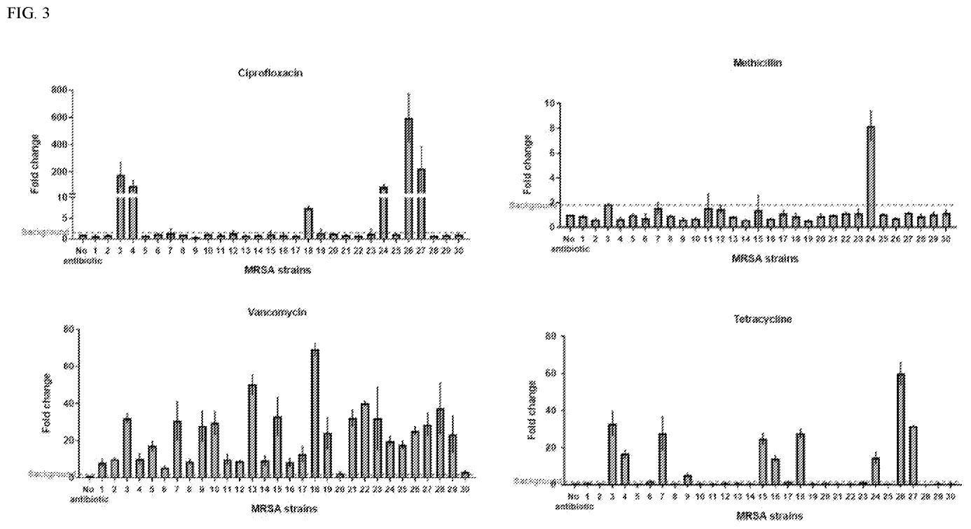

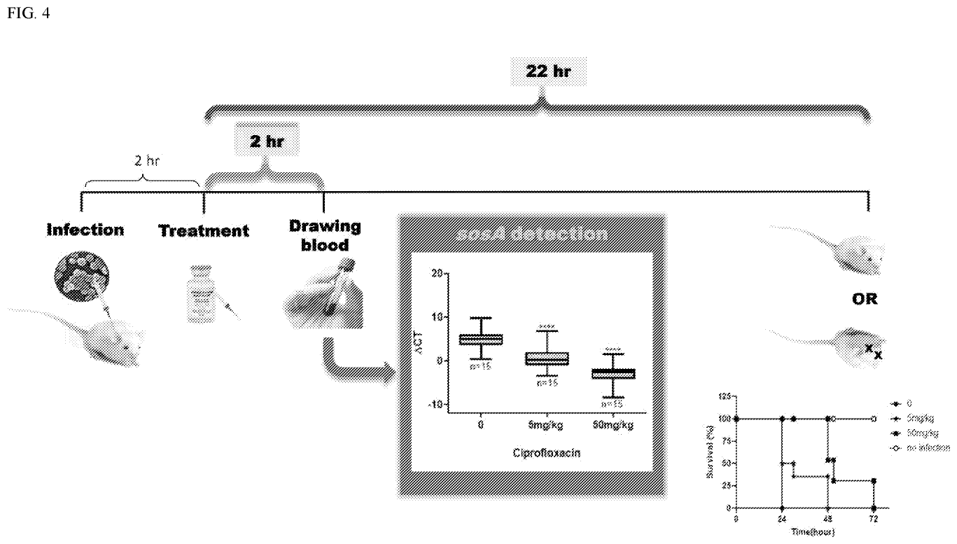

As shown in FIG. 3, the sosA expression level of methicillin-resistant S. aureus (MRSA) increased when susceptibility to antibiotics was derived and was inhibited when the resistance to antibiotics was derived, wherein the sosA expression level was similar to previously known antibiotic susceptibility/resistance properties.

As a result of comparing susceptibility/resistance diagnosis rate for types of antibiotics in Staphylococcus aureus in comparison with existing susceptibility/resistance properties using the result of analyzing the sosA expression level of 20 types of methicillin-sensitive S. aureus (MSSA) and 30 types of methicillin-resistant S. aureus (MRSA), as shown in Table 4 below, the susceptibility/resistance diagnosis rate was 98% to ciprofloxacin, 100% to vancomycin, 96% to methicillin, and 98% to tetracycline.

TABLE 4

| Types of antibiotics | Diagnostic reliability (%) |
|---|---|
| Ciprofloxacin | 98 |
| Vancomycin | 100 |
| Methicillin | 96 |
| Tetracycline | 98 |

The results demonstrate that the sosA expression levels may be used as a means to diagnose susceptibility/resistance of Staphylococcus aureus to various types of antibiotics.

### Example 3. Prediction of Treatment Prognosis According to sosA Expression

Using an animal model infected with Staphylococcus aureus, analysis was performed on the sosA expression level of Staphylococcus aureus in the blood of infected animals after antibiotic treatment, and at the same time, the prognosis of animals following treatment was monitored, so as to evaluate that the sosA expression is able to quickly predict the prognosis of treatment.

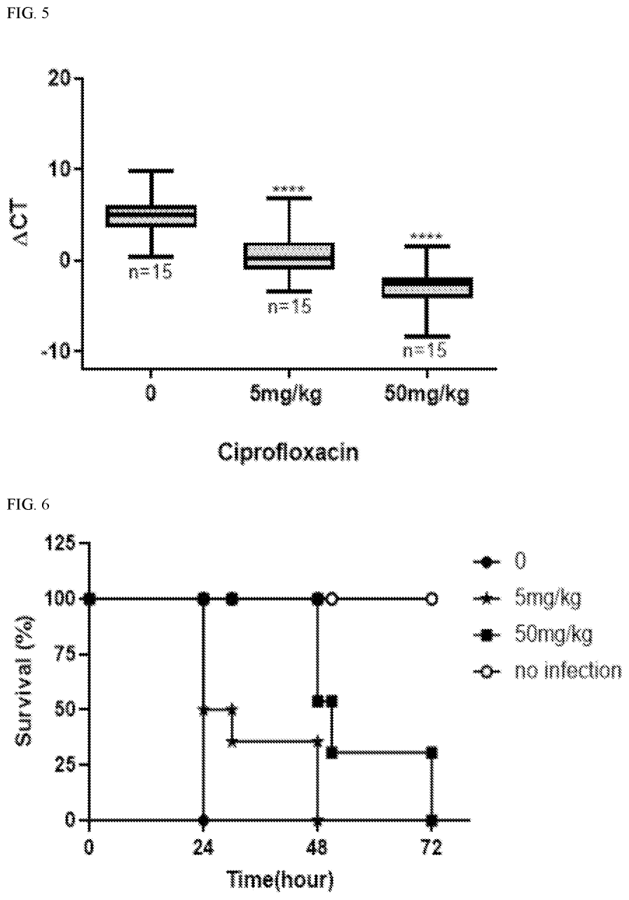

First, 2 hours later after infecting the animal model with Staphylococcus aureus, ciprofloxacin was administered at 0, 5, and 50 mg/kg as a treatment agent, and then another 2 hours later, sosA expressed by Staphylococcus aureus in the animal's blood was measured (FIG. 5). Thereafter, survival rate of the animal model was measured after 24, 48, and 72 hours (FIG. 6).

As shown in FIGS. 5 and 6, it was found that the clinical prognosis of the animal was improved as the sosA expression increased, thereby proving that sosA expression may be a marker of prognostic diagnosis.

### Example 4. Evaluation on Antibiotic Susceptibility/Resistance Using SosA Antibodies

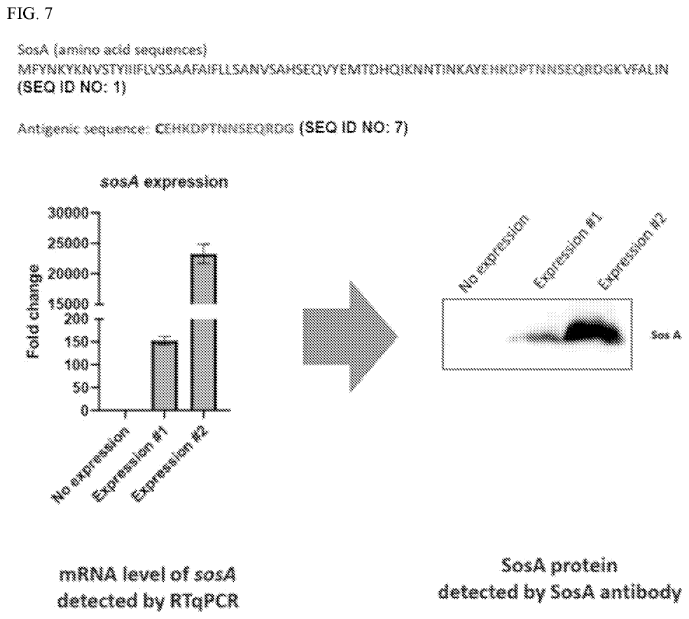

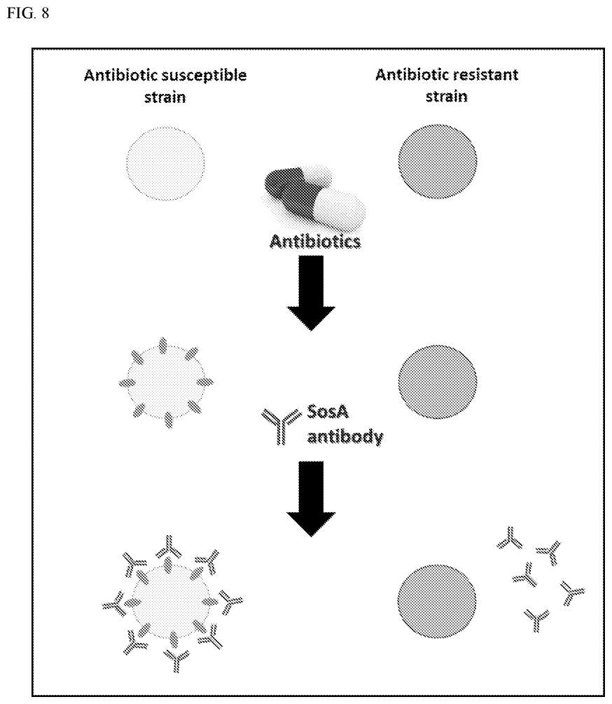

Based on the results in Example 2, Staphylococcus aureus exhibiting susceptibility to antibiotics showed the increased expression level of sosA in the presence of antibiotics, but Staphylococcus aureus exhibiting resistance to antibiotics does not express sosA. The SosA protein, encoded by the sosA gene in Staphylococcus aureus, is expressed on the surface of the bacterium. Based on these facts, an antibody that specifically binds to the SosA protein was developed (FIG. 7), and an evaluation was performed to detect susceptibility/resistance to antibiotics by detecting Staphylococcus aureus that expresses the SosA protein from a biological sample using the same.

First, antibodies that specifically bind to the SosA protein were developed. Specifically, the antibody specifically binds to an antigen represented by SEQ ID NO: 7, and a method of preparing the antibody is as follows. The antigen was prepared by synthesizing some sequences of the SosA protein in the form of peptides. Keyhole-limpet hemocyanin (KLH) was conjugated to the peptide sequence to increase the yield of antibody formation against the antigen, and to this end, cysteine (C) was added to the amino group of the synthesized antigen peptide to facilitate binding with KLH. The peptide antigen bound to the finally obtained KLH was injected into mice 7-8 times for two months. Antiserum was collected from mice induced with an immune response by antigen to check whether antibodies were formed, and at the same time, the spleen of the mice was removed, separated into single cells which were then combined with myeloma cells to prepare hybridoma cells and obtain monoclonal antibodies.

A total of seven types of hybridoma cells that form SosA-specific antibodies were collected, and each antibody obtained therefrom was named 20B5B10, 20G2H3, 22E5C11, 27D9B11, 31H3E10, 39A6C1, and 40B4B11. The sequence of each antibody was determined by an amino acid sequence converted based on the gene sequence obtained by modifying mRNA extracted from the hybridoma to cDNA.

Using the same, it was found that the SosA protein level measured thereby was similar to the expression level of the sosA gene detected by RT-PCR (FIG. 7). SosA antibodies were treated without performing a pretreatment process such as lysis for Staphylococcus aureus in which SosA is expressed to identify whether the SosA protein was detected through fluorescence analysis.

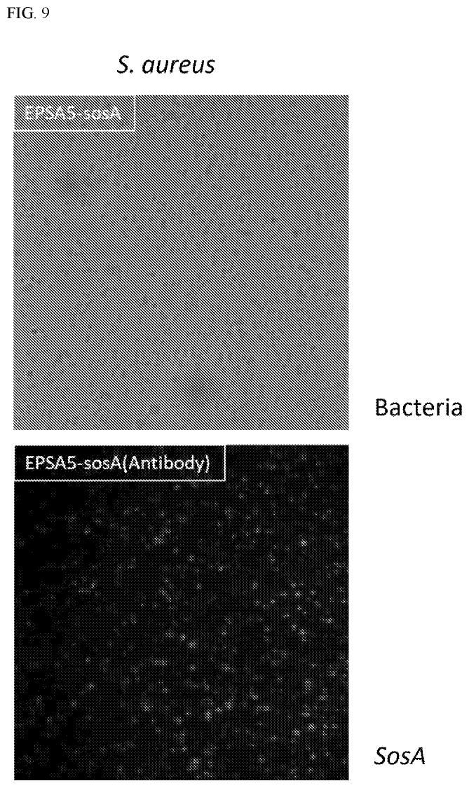

As shown in FIG. 9, as a result of identifying the presence of antibiotic-susceptible Staphylococcus aureus under an optical microscope and fluorescence-analyzing the SosA antibody, fluorescence appeared at the same site as that of antibiotic-susceptible Staphylococcus aureus.

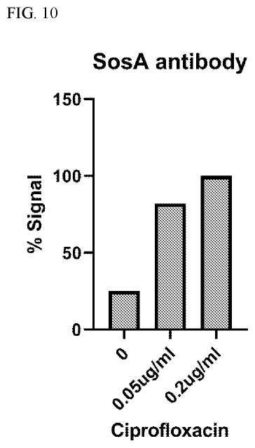

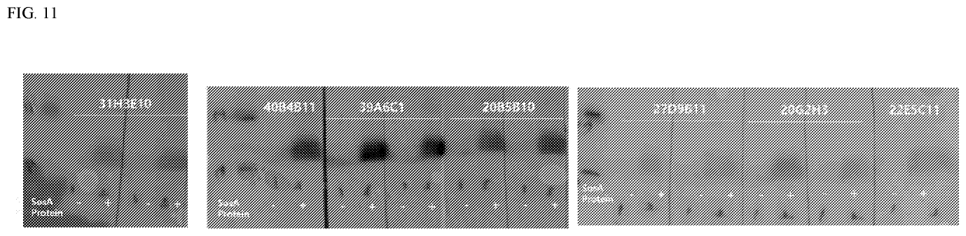

In addition, as shown in FIG. 10, expression of SosA, whose expression increases with the increase in the administration concentration of ciprofloxacin, could be detected using the antibody. In addition, as shown in FIG. 11, as a result of performing Western blot through an antigen-antibody reaction to SosA of each of the seven types of detected antibodies, it was proved that the expression of SosA may be observed using each antibody.

The above results demonstrate that it is possible to quickly evaluate antibiotic susceptibility/resistance of Staphylococcus aureus present in the sample without pretreatment of bacteria using antibodies that specifically bind to the SosA protein.

As described above, a specific part of the content of the present disclosure is described in detail, for those of ordinary skill in the art, it is clear that the specific description is only a preferred embodiment, and the scope of the present disclosure is not limited thereby. In other words, the substantial scope of the present disclosure may be defined by the appended claims and their equivalents.

1. A composition for diagnosing susceptibility of Staphylococcus aureus to an antibiotic or predicting prognosis of antibiotic treatment for infection with Staphylococcus aureus, comprising a preparation for measuring an mRNA expression level of a sosA gene or a preparation for measuring an expression level of a SosA protein.

2. The composition of claim 1, wherein the antibiotic is ciprofloxacin, vancomycin, methicillin, tetracycline, amikacin, kanamycin, gentamicin, streptomycin, doxycycline, tigecycline, erythromycin, azithromycin, clarithromycin, clindamycin, linezolid, rifampicin, levofloxacin, or moxifloxacin.

3. The composition of claim 1, wherein the preparation for measuring the mRNA expression level of the sosA gene is an antisense oligonucleotide, primer pair, or probe that specifically binds to an mRNA of the sosA.

4. The composition of claim 1, wherein the preparation for measuring the expression level of the SosA protein is an oligopeptide, monoclonal antibody, polyclonal antibody, chimeric antibody, ligand, peptide nucleic acid (PNA), or aptamer that specifically binds to the SosA protein.

5-7. (canceled)

8. A method of diagnosing susceptibility of Staphylococcus aureus to an antibiotic, the method comprising:
detecting Staphylococcus aureus having a sosA gene or a SosA protein expressed by treating a biological sample isolated from a patient infected with Staphylococcus aureus with the antibiotic and a preparation for measuring an mRNA expression level of the sosA gene or an expression level of the SosA protein; and
determining that the Staphylococcus aureus having the sosA gene or the SosA protein expressed has susceptibility to the antibiotic.

9. The method of claim 8, wherein the antibiotic is ciprofloxacin, vancomycin, methicillin, tetracycline, amikacin, kanamycin, gentamicin, streptomycin, doxycycline, tigecycline, erythromycin, azithromycin, clarithromycin, clindamycin, linezolid, rifampicin, levofloxacin, or moxifloxacin.

10. The method of claim 8, wherein the preparation for measuring the expression level of the SosA protein is an antibody, and the antibody specifically binding to the SosA protein binds to an antigen having an amino acid sequence represented by SEQ ID NO: 7.

11. The method of claim 8, wherein the detecting of Staphylococcus aureus having the SosA protein expressed on its surface comprises development of the antibody specifically binding to the SosA protein and further treating with a fluorescent material conjugated with a secondary antibody that specifically binds to the antibody.

12. The method of claim 8, wherein the fluorescent material is a green fluorescent protein (GFP), yellow fluorescent protein (YFP), blue fluorescent protein (BFP), or red fluorescent protein (RFP) and any other biologically usable fluorescent material.

13. (canceled)

14. The method of claim 844, wherein the mRNA expression level of the sosA gene is measured using reverse transcriptase polymerase reaction (RT-PCR), competitive RT-PCR, real time quantitative RT-PCR, RNase protection method, Northern blotting, or DNA chip technology.

15. The method of claim 8, wherein the expression level of the SosA protein is measured using Western blotting, enzyme linked immunosorbent assay (ELISA), radioimmunoassay (RIA), radial immunodiffusion, Ouchterlony immunodiffusion, rocket immunoelectrophoresis, immunohistochemical staining, immunoprecipitation assay, complement fixation assay, immunofluorescence, immunochromatography, fluorescence activated cell sorter (FACS) analysis, or protein chip technology.

16-17. (canceled)

18. An antibody for diagnosing susceptibility of Staphylococcus aureus to an antibiotic, wherein the antibody specifically binds to a SosA protein.

19. The antibody of claim 18, wherein the antibody comprises a heavy chain having an amino acid sequence represented by SEQ ID NO: 8 and a light chain having an amino acid sequence represented by SEQ ID NO: 9.

20. The antibody of claim 18, wherein the antibody comprises a heavy chain having an amino acid sequence represented by SEQ ID NO: 10 and a light chain having an amino acid sequence represented by SEQ ID NO: 11.

21. The antibody of claim 18, wherein the antibody comprises a heavy chain having an amino acid sequence represented by SEQ ID NO: 12 and a light chain having an amino acid sequence represented by SEQ ID NO: 13.

22. The antibody of claim 18, wherein the antibody comprises a heavy chain having an amino acid sequence represented by SEQ ID NO: 14 and a light chain having an amino acid sequence represented by SEQ ID NO: 15.

23. The antibody of claim 18, wherein the antibody comprises a heavy chain having an amino acid sequence represented by SEQ ID NO: 16 and a light chain having an amino acid sequence represented by SEQ ID NO: 17.

24. The antibody of claim 18, wherein the antibody comprises a heavy chain having an amino acid sequence represented by SEQ ID NO: 18 and a light chain having an amino acid sequence represented by SEQ ID NO: 19.

25. The antibody of claim 18, wherein the antibody comprises a heavy chain having an amino acid sequence represented by SEQ ID NO: 20 and a light chain having an amino acid sequence represented by SEQ ID NO: 21.


SEQUENCE LISTING (21 sequences)

SEQ ID NO: 1 - protein, 77 aa, Artificial Sequence
  SosA p
MFYNKYKNVSTYIIIFLVSSAAFAIFLLSANVSAHSEQVYEMTDHQIKNNTINKAYEHKDPTNNSEQRDGKVFALIN

SEQ ID NO: 2 - DNA, 234 bp, Artificial Sequence
  sosA g
atgttttacaataaatataaaaacgtatcaacatatatcatcatatttttagtttcaagtgcagcctttgcaatattcttgttaagtgcgaacgttagtgctcactcggaacaagtgtacgaaatgactgaccatcaaattaagaacaatacgataaataaagcatacgaacataaagaccctacaaacaatagcgaacaaagagatgggaaagtgttcgctttaataaattga

SEQ ID NO: 3 - DNA, 20 bp, Artificial Sequence
  sosA-f
cggaacaagtgtacgaaatg

SEQ ID NO: 4 - DNA, 20 bp, Artificial Sequence
  sosA-r
gatgattgaaggggaaatgg

SEQ ID NO: 5 - DNA, 20 bp, Artificial Sequence
  tRNA-f
gatgattgaaggggaaatgg

SEQ ID NO: 6 - DNA, 20 bp, Artificial Sequence
  tRNA-r
ggtgtcgcaactttttcaag

SEQ ID NO: 7 - protein, 15 aa, Artificial Sequence
  antigen
CEHKDPTNNSEQRDG

SEQ ID NO: 8 - protein, 463 aa, Artificial Sequence
  20B5B10-HC
MKCSWVIFFLMAVVIGINSEVQLQQSGAELVRSGASVSLSCTTSGFNIKDYYIYWVKQSPEQGLDWIGWIDPEKGDTKYAPKFQGKATLTADTSSNTAYLQLNSLTSEDTAVYFCNAPPNTGYDAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 9 - protein, 239 aa, Artificial Sequence
  20B5B10-LC
MDSQAQVLILLLLWVSGTCGDIVMSQSPSSLAVSAGEKVIMSCKSSQSLLNSRTRKNYLAWYQQKPGQSPKLLIYWAFTRASGVPDRFTGSGSGTDFTLTISSVQAEDLAVYYCKQSYNLVTFGAGTKLELIRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 10 - protein, 462 aa, Artificial Sequence
  20G2H3-HC
MKCSWVIFFLMAVVIGINSEVQLQQSGAELVRSGASVKLSCTASGLNIKDYYMYWVKQRPEQGLEWIGWIDPENGNTKYAPKFQGRATMTADTSSNTAYLQLSSLTSEDTAVYYCNAPPNNKITMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 11 - protein, 239 aa, Artificial Sequence
  20G2H3-LC
MDSQAQVLILLLLWVSGTCGDIVMSQSPSSLAVSAGEKVTMSCKSSQSLFNSRTRKNYLAWYQQKPGQSPKLLIYWASTRESGVPDRFTGSGSGTDFTLTISSVQAEDLAVYYCKQSYNLLTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 462 aa, Artificial Sequence
  22E5C11-HC
MKCSWVIFFLMAVVIGINSEVQLQQSGAELVRSGASVKLSCTSSGFNIKDFYLYWVKQRPEQGLEWIGWIDPENGNSKYAPKFQGKATMTADTSSNTAYLQLSSLTSEDTAVYYCNAPPNNKFTLDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 13 - protein, 239 aa, Artificial Sequence
  22E5C11-LC
MDSQAQVLILLLLWVSGTCGDIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLVTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - protein, 462 aa, Artificial Sequence
  27D9B11-HC
MKCSWVIFFLMAVVIGINSEVQLQQSGAELVRSGASVKLSCTTSGFNIKDYYLYWVKQRPEQGLEWIGWIDPENGNTKYAPKFQGKATMTADTSSNTAYLQLSSLTSEDTAVYYCNAPPNNKFTLDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 15 - protein, 239 aa, Artificial Sequence; identical to SEQ ID NO: 13
  27D9B11-LC

SEQ ID NO: 16 - protein, 466 aa, Artificial Sequence
  31H3E10-HC
MKCSWVIFFLMAVVIGINSEVQLQQSGAELVRSGASVKLSCTASGFNIKDHYIYWVKQRPEQGLEWIGWIDPENGKTKYGPKFQGKATLTAETSSNTAYLQLSSLTSEDTAVYYCNAPPNYTYGGGYGTDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 17 - protein, 239 aa, Artificial Sequence
  31H3E10-LC
MDSQAQVLILLLLWVSGTCGDIVMSQSPSSLAVSAGEKVTMRCKSSQSLFNSRTRKNYLAWYQQKPGQSPKLLIYWASTRESGVPDRFTGSGSGTDFTLTISSVQAEDLAVYYCKQSYDLPTFGGGTKLEFKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 18 - protein, 464 aa, Artificial Sequence
  39A6C1-HC
MKCSWVIFFLMAVVIGINSEVQLQQSGAELVRSGASVKLSCTASGFNIKDHYMYWMKQRPEQGLEWIGWIDPENGNTKYAPKFQGKATMTADTSSNTAYLQLSSLTSEDTAVYYCNAPPNSMTTSDFDYWGQGTSLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 19 - protein, 239 aa, Artificial Sequence
  39A6C1-LC
MDSQAQVLILLLLWVSGTCGDIVMSQSPSSLAVSAGEKVTMRCKSSQSLLNSRTRKNYLAWYQQKPGQSPKLLIYWASTRKSGVPDRFTGSGSGTDFTLTISRVQAEDLAVYYCKQSYNLVTFGAGTNLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 20 - protein, 464 aa, Artificial Sequence
  40B4B11-HC
MKCSWVIFFLMAVVIGINSEVQLQQSGAELVRSGASVKLSCTASGFNIKDHYMYWVKQRPEQGLEWIGWIDPENGNTKHAPKFQGKATMTADTSSNTAYLQLTSLTSEDTAVYYCNAPPNLITASGFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 21 - protein, 239 aa, Artificial Sequence
  40B4B11-LC
MDSQAQVLILLLLWVSGTCGDIVMSQSPSSLAVSAGEKVTMRCKSSQSLLNSRTRKNYLAWYQQKPGQSPKLLIYWASTRGSGVPDRFTGSGSGTDFTLTISSVQAEDLAVYYCKQSYNLVTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC